(19) United States
(12) Patent Application Publication
(10) Pub. No.: US 2004/0010312 A1
(43) Pub. Date: Jan. 15, 2004

(54) Intervertebral prosthesis

(76) Inventor: Albert Enayati, Paramus, NJ (US)

Correspondence Address: Michael G. Petit, P. O. Box 91929, Santa Barbara, CA, 93190-1929, US

(21) Appl. No.: 10/193,331

(22) Filed: Jul. 9, 2002

(51) Int. Cl. A61F 2/44

(52) U.S. Cl. 623/17.11

(57) ABSTRACT

An expandable intervertebral prosthesis includes a bone graft implant member dimensioned for insertion within an intervertebral space defined between adjacent vertebrae, thereafter adapted to vertically elevate and expand a plurality of barbs into the surrounding bone. The expandable intervertebral prosthesis has a tubular outer body portion having an axial bore with an enlarged proximal end and an exterior surface dimensioned to fit snugly within the space, and a barbed expansion cylinder slidably or rotatably mounted within the axial bore. The tubular outer body portion of the expandable intervertebral prosthesis has a plurality of longitudinal slots or holes in the wall thereof to allow the expansion and retraction of the expansion cylinder's barbs into or out of the surrounding bone. The barbs on the expansion cylinder may be elastically deformed from a normal, retracted configuration to a locking, splayed configuration wherein the outer ends of the barbs extend outwardly through the slots and exterior surface of tubular outer body to penetrate the surrounding bone as the expansion cylinder is moved. The expansion cylinder and, in one embodiment, the exterior surface of the tubular outer body portion, have a plurality of barbs disposed in circumferentially spaced relation about the body and positioned in various angles and positions respect to the axial bore. In another embodiment, the intervertebral prosthesis includes an elevating cylinder rotatably mounted within a frangible tubular outer body portion. The elevating cylinder has one or more detent positions that expand and vertically elevate the frangible tubular outer body portion of the intervertebral prosthesis body upon rotation thereof.

### BACKGROUND OF THE INVENTION

1. Field of the Invention

The present invention relates to an osteogenic interbody fusion implant device and, more particularly, to a non-threaded intervertebral bone implant having a plurality of expandable barbs configured to facilitate securement of the implant within the intervertebral space.

2. Prior Art

The spine is a flexible column formed of a plurality of bones called vertebra. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibro-cartilaginous bodies.

The intervertebral fibro-cartilages are also known as intervertebral disks and are made of a fibrous ring filled with pulpy material. The disks function as spinal shock absorbers and also cooperate with synovial joints to facilitate movement and maintain flexibility of the spine. When one or more disks degenerate through accident or disease, nerves passing near the affected area may be compressed and are consequently irritated. The result may be chronic and/or debilitating back pain. Various methods and apparatus, both surgical and non-surgical, have been designed to relieve such back pain.

One method, interbody fusion, involves stretching the spine into a natural position so that nerve root canal sizes are increased and nerve irritation is eliminated or reduced. The space between vertebrae is maintained by fusing the vertebrae in the affected area together at a fixed distance. Numerous prosthetic implants have been suggested to fill the void between vertebrae. For example, U.S. Pat. No. 4,936,848 describes a spherical cage implant made of metal or ceramics, which is inserted between adjacent vertebrae. The cage has an interior cavity within which bone fragments are inserted. Such bone fragments may be autogenic and are intended to promote subsequent bone growth and fusion of the vertebrae.

Another method of preventing contact of vertebrae is described in U.S. Pat. No. 5,011,484, wherein a stud-shaped insert is inserted longitudinally between two vertebrae and secured in position. U.S. Pat. No. 4,309,777 describes an artificial intervertebral disc having upper and lower discs, which are connected to each other by springs. The artificial disc is held in between adjacent vertebrae by spikes which project from the disc into the surface of the vertebrae in contact therewith. U.S. Pat. No. 4,743,256 describes a rigid, porous plug which can be inserted between vertebrae and held in place by prongs or screws. The porous nature of the plug is alleged to facilitate ingrowth of bone tissue.

An implantable bone plug for insertion between vertebrae is also described in U.S. Pat. No. 4,878,915, wherein, in one embodiment, the exterior of the plug is provided with external threading which will, when the plug is rotated, advance the plug into prepared sites between the vertebrae. A portion of the plug is provided with a slot designed to receive the end of a key, which is used to rotate the plug. U.S. Pat. No. 5,105,255 describes a method for forming a bored hole between two adjacent vertebrae and then inserting a graft medium such as finely chopped cortical or cancellous bone chips into the bored hole.

U.S. Pat. No. 4,961,740 is directed to a substantially open fusion cage, which is inserted between the opposing bony surfaces of adjacent vertebrae by screwing the cage into place. The cage may be filled with bone chips or other bone growth-inducing (osteogenic) substances and, when inserted into the intervertebral space, intimate contact between the bone inducing substance contained within the cage and the native bone occurs through the outer surface of the cage.

Ideally, a fusion graft should stabilize the intervertebral space and become fused to adjacent vertebrae. Moreover, during the time it takes for fusion to occur, the graft should have sufficient structural integrity to withstand the stress of maintaining the space without substantially degrading or deforming and have sufficient stability to remain securely in place prior to actual bone ingrowth fusion. Consequently, a fusion graft should contain some kind of anchor and, additionally, a bone inducing substance, which causes rapid bone growth and quick fusion of the graft to adjacent vertebrae. In addition, the material from which the fusion graft is made should be biocompatible. Further, the implant material should closely resemble host tissue and not elicit an immune response from the host.

All of the above-described implants are intended to support and maintain an appropriate intervertebral space. Unfortunately, most prior art implants do not fulfill one or more of these criteria for an ideal interbody fusion graft. For example, many of the implants, such as the one described in U.S. Pat. No. 4,936,848 are made of metals and ceramics and, while biocompatible, do not precisely mimic the body's natural bone tissue. U.S. Pat. No. 5,015,255 describes a graft in the form of bone chips that may eventually result in fusion between the vertebrae. If adequate fusion of the bone chips occurs, the final fused graft may closely mimic the body's naturally occurring tissues. However, when the bone chips are inserted, they are unconfined and may not remain contained between the vertebrae for a sufficient time to adequately fuse to each other and to adjacent vertebrae. The bone plug disclosed in U.S. Pat. No. 4,878,915 has a threaded outer surface to assist in placement of the implant between the adjacent vertebrae. The external threads, however, compromise the strength of the implant. In addition, the threaded bone implant may have a tendency of backing out of the prepared bore.

In U.S. Pat. Nos. 4,580,936, 4,859,128, 4,877,362, 5,030,050, 5,441,500, 5,489,210, 5,713,903, 5,968,044, 5,417,712, 5,501,695, 5,522,845, 5,571,104 and 6,290,701 there are disclosed a variety of anchors for attaching suture, bone and/or soft tissue to bone. The foregoing patents further disclose a number of installation tools for deploying the anchors disclosed therein. Complete details of the construction and operation of these anchors and their associated installation tools are provided in the above-identified patents, which patents are hereby incorporated herein by reference. Other prior art bone-engaging substrate fastening means often employ several straight or curved cantilevered barbs, where the barbs may be elastically deformed to permit insertion into a hole drilled in a bone. These fasteners are well known in medical applications wherein the need for high holding strength has lead to the development of anchors having multiple cantilevered barbs. In each case, the body, the attachment means, and the bone-engaging means mechanically cooperate with one another to fasten a suture, bone portion, soft tissue, prosthesis, post or other substrate to a bone.

There remains a need for improved intervertebral fusion implants with anchoring means, which more closely embody the ideal properties of a spinal fusion implant. In particular, there remains a need for an expandable intervertebral prosthesis capable of elevating the intervertebral spacing by rotation of the expansion cylinder. The ability of the prosthesis to control intervertebral elevation positions the tubular outer body of the expandable intervertebral prosthesis snugly between the vertebrae, pressing against the bone surfaces of the adjacent vertebra to promote fast bone growth and healing.

There further remains a need for an expandable intervertebral prosthesis for facilitating arthrodesis in the disc space between adjacent vertebrae with predictable and controllable initial anchorage strength sufficient to permit gradual load sharing and provide full repair and restoration of function during bone fusion. There exists a further need for a expandable intervertebral prosthesis device having elastically deformable expansion barbs on its exterior surface, wherein the outer ends of the barbs extend outwardly from the prosthetic body toward a surrounding bone when the prosthetic body, or a portion thereof, is controllably moved. There exists a further need for a expandable intervertebral prosthesis device having a movable expansion cylinder, wherein the outer ends of the barbs extend outwardly from the prosthetic body toward a surrounding bone thereafter to easily, rapidly and reliably anchor the prosthesis to the bone as the expansion cylinder is retracted from a fully extended position.

### SUMMARY

An expandable intervertebral prosthesis for implantation within a hole drilled between adjacent vertebrae, thereafter promoting the fusion of the adjacent vertebrae to one another. In a first embodiment, the intervertebral prosthesis comprises: (a) a tubular outer body portion having a proximal end, a distal end and an axial bore therebetween; and (b) an expansion cylinder slidably mounted within the axial bore of the tubular outer body portion. The tubular outer body portion has a generally cylindrical outer surface with a plurality of apertures therewithin. The tubular outer body portion may further include a plurality of elastically deformable barbs on its exterior surface that may be elastically deformed from their normally outward projecting configuration. The expansion cylinder includes a plurality barbs located in circumferentially spaced relation on the outer surface of the cylinder and disposed in various angles and attitudes with respect to the longitudinal axis. When the expansion cylinder is advanced into the axial bore of the tubular outer body portion, the barbs deform to lie within slots on the outer surface thereof. The assembly comprising the tubular outer body portion and the expansion cylinder slidably mounted within the axial bore therof comprises a first embodiment of the intervertebral prosthesis.

In operation, a hole is drilled between adjacent vertebrae and the above-described assembly (i.e., the intervertebral prosthesis) is inserted into the hole. The expansion cylinder is then partially retracted, thereby driving the outwardly biased elastically deformable barbs through the holes in the outer surface of the tubular outer body portion and into the surrounding bone, thereby anchoring the prosthesis within the intervertebral space. This embodiment of the present invention is not elevatable.

In another embodiment, the tubular outer body portion is frangible—being formed from two mirror image hemicylinders attached together along the length thereof to form a frangible joint therebetween. The frangible tubular outer body portion has an axial bore and preferably a plurality of elastically deformable barbs on the outer surface thereof. An elevating cylinder having longitudinal flanges or ridges on the outer surface thereof is rotatably disposed within the axial bore of the tubular outer body portion. The longitudinal ridges on the elevating cylinder fit snugly into a mating set of longitudinal channels or grooves on the inner wall of the axial bore of the tubular outer body portion.

In operation, a hole is drilled between adjacent vertebrae and the frangible tubular outer body portion containing the elevating cylinder is inserted into the hole. The barbs, being elastically deformable, flatten out during insertion and expand into the surrounding bone when the prosthesis is partially retracted. The elevating cylinder is then rotated through a 90° angle. As the flanges move out of the mating grooves on the inner surface of the axial bore, the flanges urge the hemicylinders apart thereby breaking the frangible joint therebetween and elevating the opposing hemicylinders to press tightly against the surrounding bone, forcing the barbs even deeper into the bone. When the 90° rotation is complete, the flanges engage a second, shallower set of grooves within the axial bore that serve as a detent position. The elevating cylinder may further include an axial bore that contains a bone graft material and a plurality of holes in the outer surface thereof.

In yet a further embodiment of the intervertebral prosthesis of the present invention, a longitudinally frangible, tubular outer body portion has an elevating cylinder rotatable mounted within the axial bore thereof, and further includes a barbed expansion cylinder slidably mounted within a second axial bore in the elevating cylinder. In operation, a hole is drilled between the adjacent vertebrae to be fused and the prosthesis is inserted into the hole. Rotation of the elevating cylinder through a 90° angle separates the hemicylinders comprising the tubular outer body portion, forcing the opposing surfaces thereof against the surrounding bone, After rotation of the elevating cylinder is complete, partial retraction of the expansion cylinder drives the barbs on the surface thereof through holes in the elevating cylinder and tubular outer body portion and into the bone to anchor the prosthesis within the hole. In all embodiments, the elevating cylinder and/or the expansion cylinder may include a bone graft material housed within an axial bore therewithin.

In yet a further embodiment of an intervertebral prosthesis in accordance with the present invention, the prosthesis comprises a single tubular outer body portion having a plurality of holes and barbs on the outer cylindrical surface thereof and an axial bore. The barbs are elastically deformable. The plurality of holes in the surface thereof extend inwardly to the axial bore. The axial bore contains a bone graft material. In operation, a hole is drilled between adjacent vertebrae and the tubular outer body portion is inserted into the hole and advanced thereinto. As the prosthesis is advanced, the barbs bend, lying against the surface of the prosthesis. When the prosthesis is fully inserted into the hole, retraction of the prosthesis drives the elastically deformable barbs into the surrounding bone thereby anchoring the prosthesis within the hole. The plurality of holes in the surface of the tubular outer body permit ingrowth of bone into the bone graft material housed within the axial bore thereby promoting fusion of the adjacent vertebrae.

The features of the invention believed to be novel are set forth with particularity in the appended claims. However the invention itself, both as to organization and method of operation, together with further objects and advantages thereof may be best understood by reference to the following description taken in conjunction with the accompanying drawings in which:

### BRIEF DESCRIPTION OF THE DRAWINGS

FIG. 1 is a perspective view of an intervertebral prosthesis comprising an expansion cylinder slidably and rotatably disposed within the axial bore of a tubular outer body portion in accordance with a preferred embodiment of the present invention.

FIG. 2 is a perspective view of the expansion cylinder of the intervertebral prosthesis of FIG. 1.

FIG. 2a is an end view of the expansion cylinder of FIG. 2.

FIG. 3 is a perspective view of an elevatable and expandable intervertebral prosthesis in accordance with a second preferred embodiment of the present invention wherein a frangible tubular outer body portion has an elevating cylinder rotatably disposed within the axial bore thereof.

FIG. 4 is a perspective view of an elevating cylinder suitable for use with the frangible tubular body portion as shown in the intervertebral prosthesis of FIG. 3.

FIG. 5 is a perspective view of an expansion cylinder as shown in FIG. 2 but further including a bone graft material in an axial bore thereof and a plurality of holes in the outer surface.

FIG. 5a is an end view of the expansion cylinder of FIG. 5.

FIG. 6 is a perspective view of an elevatable and expandable embodiment of an intervertebral prosthesis prior to elevation and expansion illustrating, in phantom, how the plurality of curved barbs extend outwardly from the frangible tubular outer body portion when the prosthesis is deployed within a hole drilled in or between adjacent vertebrae.

FIG. 7 is an end view of the elevatable and expandable intervertebral prosthesis of FIG. 6 prior to the elevation and expansion of the barbs.

FIG. 8 is an end view of the elevatable and expandable intervertebral prosthesis of FIG. 6 following the elevation and expansion of the barbs and illustrating the separation of the hemicylinders comprising the frangible tubular outer body portion following rotation of the elevating cylinder.

FIG. 8a is a perspective view of an embodiment of the intervertebral prosthesis of the present invention consisting of a tubular outer body portion wherein there are no expansion or elevating cylinders.

FIG. 9 is a partially cutaway elevational view of an expandable intervertebral prosthesis insertion tool operable for inserting the tubular outer body of expandable intervertebral prosthesis into a hole drilled in bone and for forcing a expansion cylinder into the axial bore of the tubular outer body.

FIG. 10 is a schematic left end view of the expandable intervertebral prosthesis insertion tool of FIG. 9.

FIG. 11 is a right end view of the expandable intervertebral prosthesis insertion tool illustrated in FIG. 9.

FIG. 12 is a side elevational view of an expansion cylinder insertion rod adapted for use with the expandable intervertebral prosthesis insertion tool of FIG. 9.

FIG. 13 is a plan view of an intervertebral prosthesis of the present invention inserted into a hole drilled between adjacent vertebrae.

### DESCRIPTION OF THE PREFERRED EMBODIMENTS

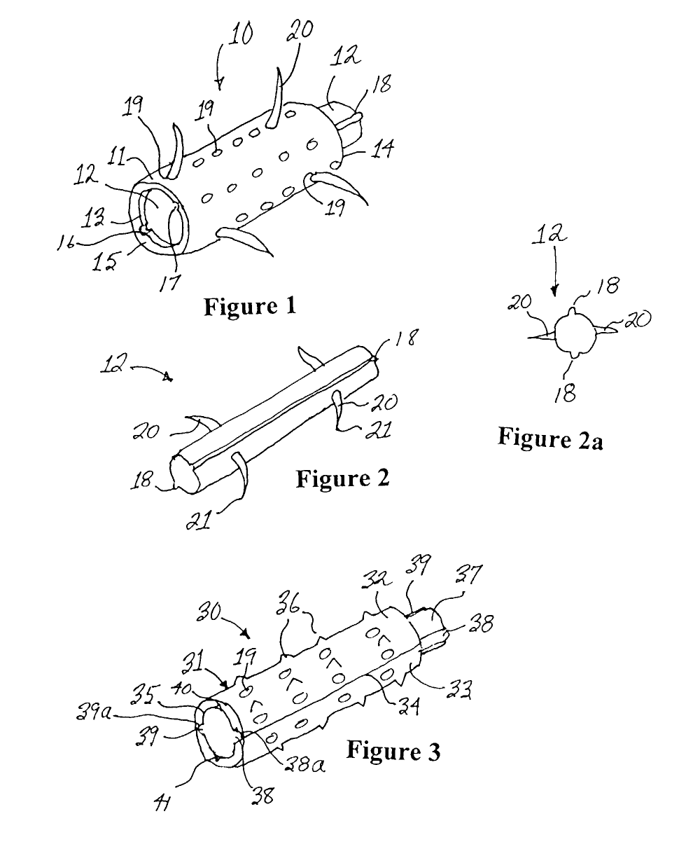

With reference to FIG. 1, the expandable intervertebral prosthesis 10 in accordance with a first preferred embodiment of the present invention comprises a tubular outer body portion 11 with an expansion cylinder 12 slidably disposed within an axial bore 13 in the tubular outer body portion 11. The expandable intervertebral prosthesis 10 has a proximal end 14 and a distal end 15. The wall of the tubular outer body portion has a plurality of holes 19 therein. The cylindrical axial bore 13 is coextensive with the length of the tubular outer body portion 11. The expansion cylinder 12 having a guide track 18 and a plurality of elastically deformable barbs 20 disposed along the length thereof is shown in greater detail in FIG. 2.

In order to use the embodiment of the expandable intervertebral prosthesis indicated at numeral 10, a hole is first drilled between adjacent vertebrae in a direction substantially transverse to the direction of the spine, the hole being centered between adjacent vertebrae. The tubular outer body portion 11 (without barbs) is inserted into the hole. The outer diameter of the expansion cylinder 12 is dimensioned to slidably fit within the axial bore 13 of the tubular outer body portion 11 of the expandable intervertebral prosthesis 10. At least one longitudinal guiding track 16 and 17 on the interior wall of the axial bore 13 is dimensioned to fit snugly to at least one mating track 18 on the outer surface of the expansion cylinder 12. The barbs 20 on the expansion cylinder 12 are depressed by the application of external pressure to the proximal end 14 of the expansion cylinder 12 as it is slidably guided down through the axial bore 13 to the distal end 14 of the tubular outer body portion 11. As the barbed portion of the expansion cylinder enters the axial bore, barbs 20, which are formed out of an elastically deformable material, are forced radially inwardly so as to be disposed entirely within the axial bore 13 of the outer tubular member 11. When the distal end 15 of the expansion cylinder 12 is fully advanced into the axial bore 13, the sharp tips 21 of the barbs 20 are adjacent to holes 19 and partially expand thereinto. The expansion cylinder 12 is then retracted and the sharp outer ends 21 of the barbs 20 are forced progressively outwardly thereby penetrating the cancellous bone. As the expansion cylinder is progressively retracted from within the axial bore, that is, pulled in a proximal direction, the sharp outer ends 21 of the barbs 20 enter and are forced into the cortical bone. When the barbs 20 are fully expanded, no further retraction of the expansion cylinder is possible and the intervertebral prosthesis is locked in position between adjacent vertebrae.

To remove the embedded intervertebral prosthesis from the bone, a pushpin (not shown) is inserted into the proximal end of axial bore 13 to contact the proximal end of the expansion cylinder 12. When pressure is applied to the pushpin, the expansion cylinder is forced in a distal direction until the distal end of the expansion cylinder underlies the distal end of the tubular outer body portion. In this fully depressed position, the barbs 20 are retracted through the holes 19 from within the surrounding bone and folded against the outer surface of the expansion cylinder 12 to lie within the axial bore 13 in a space between the outer surface of the expansion cylinder 12 and the inner surface of the tubular outer body portion 11. The expandable intervertebral prosthesis 10 may then be removed from the hole by applying traction to the tubular outer body portion 11.

An elevatable embodiment of an intervertebral prosthesis in accordance with the present invention is shown in perspective view at numeral 30 in FIG. 3. In the elevatable embodiment 30, the tubular outer portion 31 comprises two hemicylinders 32 and 33 attached along the length thereof by frangible attachment means 34 to form a tube having an axial bore 35 coextensive with the length thereof. The outer surface of the tubular outer portion 31 preferably includes a plurality of relatively short spikes 36 projecting outwardly therefrom. When elevating cylinder 37 is rotated within the axial bore 35, camlike expansion flanges 38 and 39 on the cylindrical outer surface of the elevating cylinder are forced out of mating detent grooves 38a and 39a in the wall of the axial bore and urge the hemicylinders 32 and 33 apart, breaking the frangible connection 34 therebetween and forcing the hemicylinders against surrounding bone (not shown) until the expansion flange(s) come to rest in relatively shallow detent grooves 40 and 41 within the axial bore, thereby elevating the adjacent vertebrae upon which the opposing hemicylinders are pressed. The pressure forces spikes 36 into the surrounding bone thereby providing positive attachment of the outer tubular body 31 to the bone.

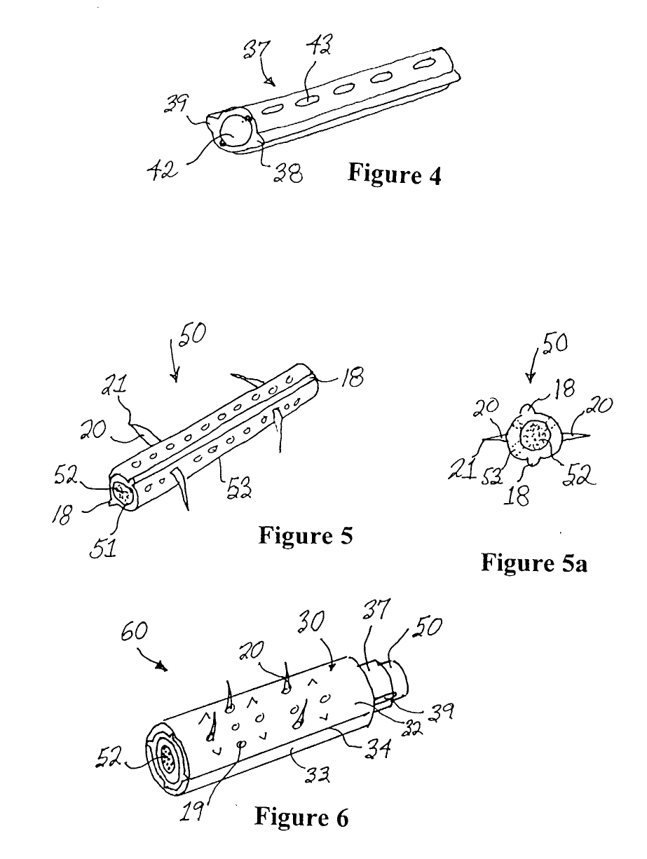

The rotatable elevating cylinder 37, shown in perspective view in FIG. 4, may, in turn, have a second axial bore 42 coextensive with the length thereof through which a barbed expansion cylinder, such as the expansion cylinder shown at 12 in FIGS. 1 and 2, may be inserted. Slots 43 in the wall of the elevating cylinder 37 accommodate the folded barbs 20 during insertion of the expansion cylinder 12 into the axial bore 42 of the elevating cylinder 37. When the expansion cylinder 12 is retracted, the barbs 20 expand through the holes 19 in the tubular outer body portion 31 and enter the surrounding bone thereby firmly anchoring the prosthesis to the bone.

In a further embodiment of an intervertebral prosthesis in accordance with either of the two foregoing embodiments, the expansion cylinder 50 may be modified by hollowing it out to provide an axial bore 51 that can be used to contain bone graft material 52 as shown in FIG. 5. The bone graft material 52 may be bone chips or a suitable osteogenic material. The expansion cylinder 50 has a plurality of holes 53 therein and an outer diameter dimensioned to be received within the axial bore 42 of elevating cylinder 37 (FIG. 4). The holes 53, together with the slots 43 in the extending cylinder, enable bone ingrowth into the core of the expansion cylinder 50.

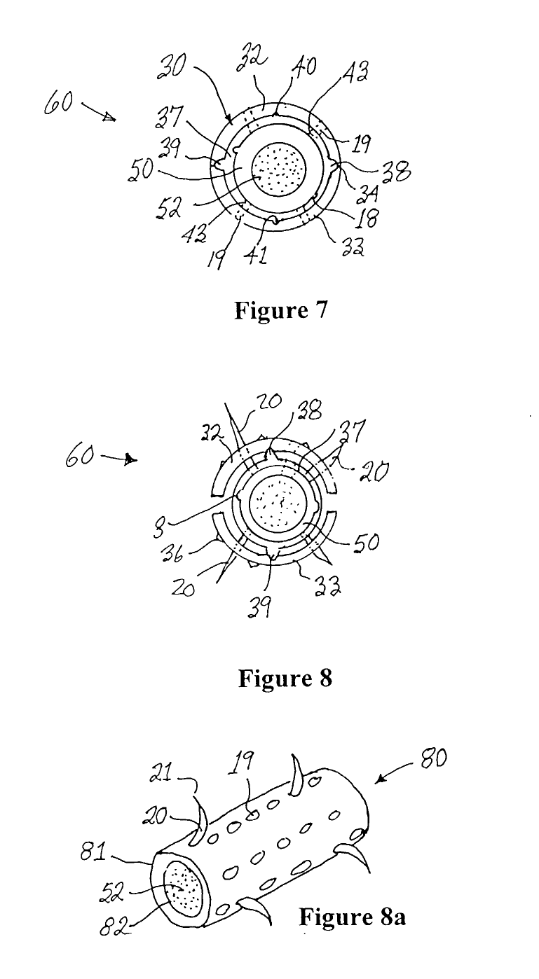

The operation of an intervertebral prosthesis comprising a frangible tubular outer body portion 30, an elevating cylinder 37 and the expansion cylinder 50 is best understood with reference to FIG. 6. In FIG. 6, an elevatable, expandable embodiment of an intervertebral prosthesis is illustrated in perspective view at numeral 60. The prosthesis 60 has an outermost diameter dimensioned to be inserted into a hole drilled between adjacent vertebrae. The prosthesis 60 includes a tubular outer body portion 30 comprising a pair of mirror-image hemicylinders 32 and 33 joined along the length thereof by a frangible joint 34. An elevating cylinder 37 having a pair of elevating flanges 39 projecting laterally from the outer surface of the elevating cylinder and coextensive with the length thereof is rotatably disposed within the axial bore of the tubular outer body portion 30. After the tubular outer body portion 30 is inserted within the hole previously drilled between adjacent vertebrae, the elevating cylinder 37 is rotated ninety degrees. During rotation, the flanges 39 are forced out of the detent grooves 38a and 39a and urge the hemicylinders 32 and 33 apart thereby breaking frangible joint 34 and pressing the outer surface of the hemicylinders comprising the tubular outer body portion against surrounding bone (not shown). When the 90° rotation is complete, a pair of detent grooves 40 and 41 (FIGS. 3 and 7) on the inner diameter of the tubular outer body portion engage the flanges 38 and 39 thereby locking the elevating cylinder in a position that creates a space between the hemicylinders as shown in FIG. 8. After the elevating cylinder is rotated and locked into position, a barbed expansion cylinder 50, slidably disposed within an axial bore 42 of the elevating cylinder 37, is partially retracted; forcing the barbs 20, which were previously disposed within the slots 43 of the elevating cylinder 37, outwardly through holes 19 and into the surrounding bone thereby anchoring the prosthesis 60 into the intervertebral hole.

FIG. 7 is an end view of the distal end of an expandable intervertebral prosthesis 60 prior to separation of the hemicylinders 32 and 33 and expansion of the barbs 20. FIG. 8 is a distal end view of the prosthesis 60 after rotation of the extending cylinder and partial retraction of the expansion cylinder to extend the elastically deformable barbs 20 into the surrounding bone (not shown).

It is preferred that the barbs 20 of expansion cylinders 12 or 50 and the spikes 36 of the tubular outer body portion 30 are formed out of polymer blends of glycolide and/or lactide homopolymer, copolymer and/or glycolide/lactide copolymer and polycaprolactone copolymers, and/or copolymers of glycolide, lactide, poly (L-lactide-co-DL-lactide), caprolactone, polyorthoesters, polydioxanone, trimethylene carbonate and/or polyethylene oxide or any other bioabsorbable material. A pseudoelastic shape memory alloy of the type disclosed in U.S. Pat. No. 4,665,906 entitled “Medical Devices Incorporating SIM Alloy Elements”, issued May 19, 1987 to Jervis, which patent is specifically incorporated herein by reference, may also be used to fabricate the barbs 20. By way of example, one such pseudoelastic shape memory alloy might be a nickel titanium alloy such as Nitinol, which is available from Flexmedics of Minneapolis, Minn., among others. The use of such a material, in combination with the normal orientation of the barbs relative to the anchor body, permits the barbs to initially deflect inwardly to the extent required to permit the tubular outer body portion to be advanced into the drilled hole, or for the expansion cylinder 12 to be advanced into the axial bore of the tubular outer body portion 11, yet resiliently “spring back” toward their normal, outwardly projecting position so as to prevent the prosthesis 10 or 60 from withdrawing from the drilled hole after being deployed therein. Other implantable (biocompatible) materials that may be used to fabricate an intervertebral prosthesis in accordance with any of the embodiments of the present invention include stainless steel, titanium and cobalt-chrome alloy.

In yet a further embodiment of an intervertebral prosthesis in accordance with the present invention, indicated generally at numeral 80 in FIG. 8a, the prosthesis 80 comprises a single tubular outer body portion 81 having a plurality of holes 19 and barbs 20 on the outer cylindrical surface thereof and an axial bore 82. The barbs 20, having sharp, outwardly biased tips 21, are elastically deformable. The plurality of holes 19 in the surface thereof extend inwardly to the axial bore 82. The axial bore 82 contains a bone graft material 52. In operation, in order to implant the intervertebral prosthesis 80, a hole is drilled between adjacent vertebrae and the tubular outer body portion 81 is inserted into the hole and advanced thereinto. As the prosthesis is advanced into the drilled hole, the (elastically deformable) barbs 20 bend, lying against the outer surface of the prosthesis 80. When the prosthesis 80 is fully inserted into the hole, retraction of the prosthesis drives the elastically deformable barbs 20 into the surrounding bone (not shown) thereby anchoring the prosthesis within the hole. The plurality of holes 19 in the surface of the tubular outer body 81 permit ingrowth of host bone into the bone graft material housed within the axial bore thereby promoting fusion of the adjacent vertebrae.

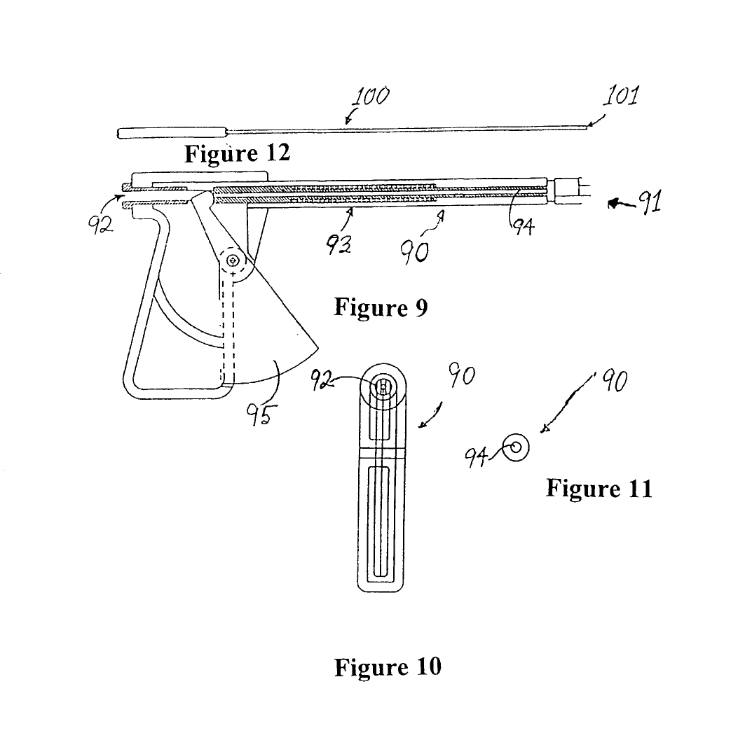

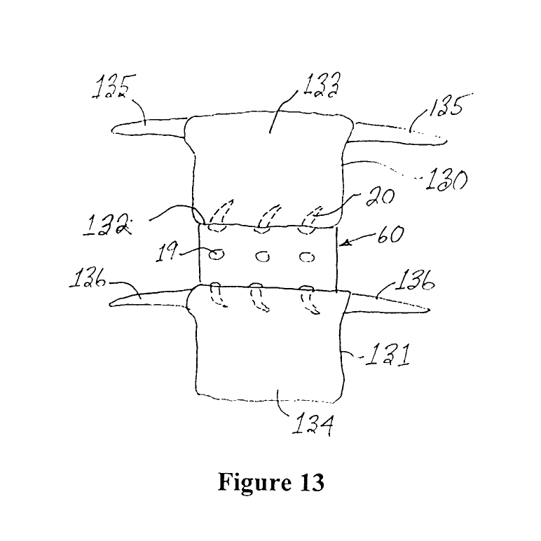

A tool useful for inserting an expandable intervertebral prosthesis 10, 60 or 80 into a hole drilled in bone in accordance with another aspect of the present invention is shown in elevational cross-sectional view at 90 in FIG. 9 and front and rear end views in FIGS. 10 and 11 respectively. The tool 90 has a distal bone fastener-grasping end 91 and a proximal end 92 and a barrel 93 there between having an axial bore 94 dimensioned to slidably accommodate the proximal end of the expansion cylinder therewithin. With alternate reference to the embodiment 60 of the expandable intervertebral prosthesis shown in FIG. 6, the proximal end of the tubular outer body 53 of the expandable intervertebral prosthesis 50 is held securely within the distal end 91 of the tool 90 by suitable bone fastener grasping means, and the opposing (distal) end of the expandable intervertebral prosthesis is inserted into a hole drilled in a bone (FIG. 13). Squeezing pivotally mounted trigger 95 forces the expansion cylinder 37 into the axial bore of the outer tubular body 30 comprising the expandable intervertebral prosthesis 60. A clutch (not shown) rotates the expansion cylinder 37 disposed within the axial bore of the outer tubular body 30 thereby elevating and separating the hemicylinders comprising tubular body 30. When the trigger 95 is released, a spring (not shown) retracts the extension cylinder 50 thereby expanding barbs 20 into the surrounding bone. The expandable intervertebral prosthesis 60 is released when the trigger 75 returns to its initial position. A pushrod 100, dimensioned to fit within the axial bore 94 of the tool barrel 93, is used for removing the prosthesis 60 from the hole. The prosthesis 60 is removed by placing the pushrod within the axial bore 94 of the tool 90 and placing the distal end 101 of the pushrod 100 against the extension pin and advancing it forward to retract the barbs. With the extension cylinder fully advanced and the barbs retracted, the expansion cylinder, if necessary, can then be rotated ninety degrees to bring the hemicylinders into juxtaposition along the length thereof, and the tubular outer body portion extracted from the hole by traction.

FIG. 13 is a plan view of an intervertebral prosthesis 60 of the present invention inserted into a hole 132 drilled between the bodies 133 and 134 of two adjacent vertebrae 130 and 131. The transverse processes 135 and 136 of vertebrae 130 and 131 are unaffected by the presence of the prosthesis 60 within the hole 132. The holes 19 enable bone growth between the vertebral bodies 133 and 134 to extend into the bone graft material 52 housed within the axial bore of the extension cylinder thereby fusing the vertebrae to one another.

While particular embodiments of the present invention have been illustrated and described, it would be obvious to those skilled in the art that various other changes and modifications can be made without departing from the spirit and scope of the invention. For example, axially elevating the expandable intervertebral prosthesis may be perform by other mean such as conical shape cylinders, screw, nail or wedge driven expander, collapsing, reducing or expanding diameter or any other expansion driven design. Other example, the outer tubular member 20 can be either expanded partially, fully or remain un-deformed when the expansion cylinder is advanced into the axial bore 21 of the outer tubular member 22 in a distal direction. Similarly, the outer surface of the outer tubular member is disclosed as cylindrical in the preferred embodiment, but may be hexagonal or have another polygonal cross sectional profile. It is therefore intended to cover in the appended claims all such changes and modifications that are within the scope of this invention.

What I claim is:

1. An intervertebral prosthesis for implantation within a hole drilled between adjacent vertebrae in the spine of an animal, thereafter enabling the adjacent vertebrae to fuse to one another, comprising: (a) an elongate tubular member having an axial bore and a cylindrical outer surface dimensioned to fit snugly within said drilled hole; (b) a bone graft material disposed within said axial bore; (c) a plurality of holes in said cylindrical outer surface extending inwardly to said axial bore; and (d) a plurality of elastically deformable barbs on said cylindrical outer surface.

2. An expandable intervertebral prosthesis for implantation within a hole drilled between adjacent vertebrae in the spine of an animal, the implanted prosthesis thereafter enabling the adjacent vertebrae to fuse to one another, the intervertebral prosthesis comprising:
(a) a tubular outer body portion having a proximal end, a distal end and an elongate body portion with a first axial bore therebetween, said tubular outer body portion having a generally cylindrical first outer surface with a plurality of first apertures therein;
(b) an elongate expansion cylinder slidably disposed within said first axial bore, said expansion cylinder having a second axial bore and a second outer cylindrical surface;
(c) a bone graft material disposed within said second axial bore; and
(d) a plurality of second holes in said second outer cylindrical surface providing a plurality of conduits between said second cylindrical outer surface and said second axial bore; and
(e) a plurality of elastically deformable barbs disposed on said second outer cylindrical surface of said expansion cylinder wherein when said expansion cylinder is partially retracted from within said first axial bore, said plurality of barbs extend outwardly through said first apertures in said cylindrical outer surface of said tubular outer body portion.

3. An elevatable intervertebral prosthesis for implantation within a hole drilled between adjacent vertebrae in the spine of an animal, the implanted prosthesis thereafter enabling the adjacent vertebrae to fuse to one another, the intervertebral prosthesis comprising:
(a) a tubular outer body portion comprising a pair of hemicylinders attached to one another along the length thereof by a frangible joint and having a proximal end, a distal end and a first axial bore therebetween, said tubular outer body portion having a generally cylindrical first outer surface with a plurality of first apertures therein and at least two longitudinal detent grooves on first said axial bore;
(b) an elongate elevating cylinder rotatably disposed within said first axial bore, said elevating cylinder having a second axial bore and a second outer cylindrical surface with at least two longitudinal flanges on said cylindrical outer surface dimensioned to releasably engage said detent grooves and;
(c) a bone graft material disposed within said second axial bore.
(d) a plurality of second holes in said second outer cylindrical surface providing a plurality of conduits between said second cylindrical outer surface and said second axial bore.

4. An expandable elevatable intervertebral prosthesis for implantation within a hole drilled between adjacent vertebrae in the spine of an animal, the implanted prosthesis thereafter enabling the adjacent vertebrae to fuse to one another, the intervertebral prosthesis comprising:
(a) a tubular outer body portion comprising a pair of hemicylinders attached to one another along the length thereof by a frangible joint and having a proximal end, a distal end and a first axial bore therebetween, said tubular outer body portion having a generally cylindrical first outer surface with a plurality of first apertures therein and at least two longitudinal detent grooves on first said axial bore;
(b) an elongate elevating cylinder rotatably disposed within said first axial bore, said elevating cylinder having a second axial bore and a second outer cylindrical surface with at least two longitudinal flanges on said cylindrical outer surface dimensioned to releasably engage said detent grooves and wherein rotation of said elevating cylinder is operable for breaking said frangible joint and separating said hemicylinders comprising said tubular outer body portion;
(c) a plurality of second holes in said second outer cylindrical surface providing a plurality of conduits between said second cylindrical outer surface and said second axial bore;
(d) an elongate expansion cylinder slidably disposed within said second axial bore, said expansion cylinder having a third axial bore and a third outer cylindrical surface;
(e) a bone graft material disposed within said third axial bore; and
(f) a plurality of third holes in said third outer cylindrical surface providing a plurality of conduits between said third cylindrical outer surface and said third axial bore; and
(g) a plurality of elastically deformable barbs disposed on said third outer cylindrical surface of said expansion cylinder wherein when said expansion cylinder is partially retracted from within said first axial bore, said plurality of barbs extend outwardly through said first apertures in said cylindrical outer surface of said tubular outer body portion.

5. The expandable intervertebral prosthesis in accordance with claim 1 wherein said expandable intervertebral prosthesis is made from a material selected from the group comprising a bioabsorbable, moldable polymer, a pseudoelastic shape memory alloy, titanium, stainless steel or a cobalt-chrome alloy.
